(19) United States
(12) Patent Application Publication
(10) Pub. No.: US 2005/0239878 A1
(43) Pub. Date: Oct. 27, 2005

(54) Inhibitors of methionine aminopeptidase-2 and uses thereof

(75) Inventors: Charles Thompson, Chalfont, PA (US); Christopher C. Arico-Muendel, West Roxbury, MA (US)

Correspondence Address: LAHIVE & COCKFIELD, LLP., 28 STATE STREET, BOSTON, MA, 02109, US

(73) Assignee: Praecis Pharmaceuticals, Inc., Waltham, MA (US)

(21) Appl. No.: 11/025,568

(22) Filed: Dec. 29, 2004

Related U.S. Application Data
(60) Provisional application No. 60/533,431, filed on Dec. 29, 2003.

(51) Int. Cl. A61K 31/336; C07D 45/02

(52) U.S. Cl. 514/475; 549/332; 549/551

(57) ABSTRACT

The instant invention provides compositions and methods for treating a subject suffering from one of a number of conditions, including an angiogenic disease, such as cancer, an autoimmune disorder or a parasitic infection.

### RELATED APPLICATIONS

This application claims priority to U.S. Provisional Application Ser. No. 60/533,431, filed Dec. 29, 2003, the entire contents of which are incorporated herein by reference.

### BACKGROUND OF THE INVENTION

Angiogenesis is the fundamental process by which new blood vessels are formed and is essential to a variety of normal body activities (such as reproduction, development and wound repair). Although the process is not completely understood, it is believed to involve a complex interplay of molecules which both stimulate and inhibit the growth of endothelial cells, the primary cells of the capillary blood vessels. Under normal conditions, these molecules appear to maintain the microvasculature in a quiescent state (i.e., one of no capillary growth) for prolonged periods which may last for as long as weeks or in some cases, decades. When necessary, however, (such as during wound repair), these same cells can undergo rapid proliferation and turnover within a 5 day period (Folkman, J. and Shing, Y., Journal of Biological Chemistry, 267(16): 10931-10934, and Folkman, J. and Klagsbrun, M. (1987) Science, 235: 442-447).

Although angiogenesis is a highly regulated process under normal conditions, many diseases (characterized as “angiogenic diseases”) are driven or characterized by persistent unregulated angiogenesis. Otherwise stated, unregulated angiogenesis may either cause a particular disease directly or exacerbate an existing pathological condition. For example, ocular neovacularization has been implicated as the most common cause of blindness and dominates approximately 20 eye diseases. In certain existing conditions such as arthritis, newly formed capillary blood vessels invade the joints and destroy cartilage. In diabetes, new capillaries formed in the retina invade the vitreous, bleed, and cause blindness. Growth and metastasis of solid tumors are also angiogenesis dependent (Folkman, J. (1986) Cancer Research 46: 467-473 and Folkman, J. (1989) Journal of the National Cancer Institute 82: 4-6). It has been shown, for example, that tumors which grow to a size greater than 2 millimeters must obtain their own blood supply and do so by inducing the growth of new capillary blood vessels. Once these new blood vessels become embedded in the tumor, they provide nutrients to the tumor and can serve as a means for tumor cells to enter the circulation and metastasize to distant sites, such as the liver, lung or bone (Weidner, N., et al. (1991) The New England Journal of Medicine 324(1):1-8).

Fumagillin is a known compound which has been used as an antimicrobial and antiprotozoal. Its physicochemical properties and method of production are well known (U.S. Pat. No. 2,803,586 and Proc. Nat. Acad. Sci. USA (1962) 48:733-735). Fumagillin and certain types of fumagillin analogs have also been reported to exhibit antiangiogenic activity. However, the use of such inhibitors (e.g., TNP-470) may be limited by their rapid metabolic degradation, erratic blood levels, and by dose-limiting central nervous system (CNS) side effects.

Accordingly, there is still a need for angiogenesis inhibitors which are more potent, less neurotoxic, more stable, and/or have longer serum half-lives.

### SUMMARY OF THE INVENTION

The present invention provides compositions and methods for treating a subject suffering from one of a number of conditions, including an angiogenic disease, such as cancer, an autoimmune disorder or a parasitic infection.

In one embodiment, the invention provides the compounds of the general formula A-B, wherein A is a group having a structure as set forth in one of Formulas I-VII below: 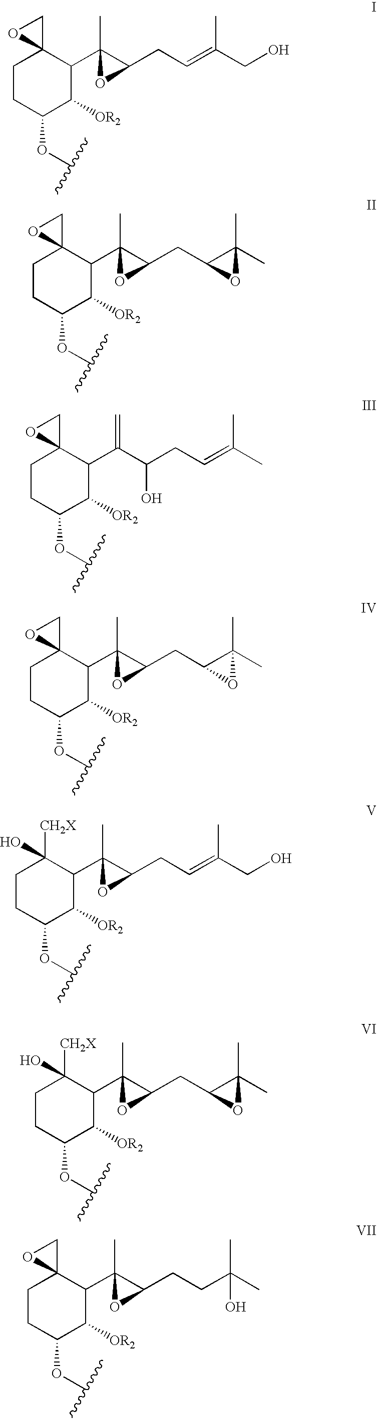
In each of Formulas I to VI, the oxygen atom at the bottom of the cyclohexane ring (i.e., in position 4 relative to the spiroepoxide in I, II<III, IV and VII, or in position 4 relative to the —CH₂X group in V and VI) is the attachment point for B and R₂ is hydrogen or C₁-C₆-alkyl, preferably methyl. In Formula VI, X is a halogen atom, a dialkylsulfinium group, a thioalkoxy group, a thioaryloxy group or another suitable leaving group. Preferably, X is bromine, chlorine or iodine. More preferably, X is chlorine.

B is an alkanoyl group, an aroyl group, an alkoxycarbonyl group, a carbamoyl group, or an N-substituted carbamoyl group.

The present invention also provides pharmaceutical compositions comprising one or more compounds of the formula A-B and a pharmaceutically acceptable carrier, and methods of using these compounds and pharmaceutical compositions for treating a variety of diseases and conditions, including angiogenic conditions, such as various cancers, lymphomas, and diseases characterized by inappropriate vascularization, and autoimmune disorders, such as rheumatoid arthritis and psoriasis

Other features and advantages of the invention will be apparent from the following detailed description and claims.

### DETAILED DESCRIPTION OF THE INVENTION

The present invention provides compounds which are inhibitors of the enzyme methionine aminopeptidase-2 (MetAP-2), pharmaceutical compositions comprising one or more of these compounds and methods for treating a subject suffering from one of a number of conditions, including angiogenic conditions and other conditions which respond to the inhibition of MetAP-2, such as cancer, including solid tumors and lymphoid malignancies, parasitic infections and autoimmune disorders.

In one embodiment, the compounds of the invention include compounds of the formula A-B wherein A is selected from the structures set forth in Formulas I-VI, above, and B is of the formula: 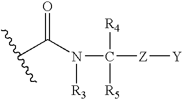
wherein R₃ is hydrogen or alkyl, preferably C₁-C₄-alkyl. R₄ and R₅ are each, independently, hydrogen, substituted or unsubstituted alkyl, substituted or unsubstituted aryl, substituted or unsubstituted arylalkyl, substituted or unsubstituted heteroaryl or substituted or unsubstituted heteroarylalkyl. Preferably, R₄ is hydrogen and R₅ is not hydrogen. Z is —C(O)— or -alkylene-C(O)—; and Y is —OR₆ or —N(R₇)R₈, wherein R₆, R₇ and R₈ are each, independently, hydrogen, substituted or unsubstituted alkyl, substituted or unsubstituted aryl or substituted or unsubstituted azacycloalkyl. R₇ and R₈ can also, together with the nitrogen atom to which they are attached, form a heterocyclic ring structure.

In a preferred embodiment, B is of the structure 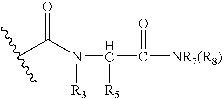
wherein R₃, R₅, R₇ and R₈ have the meanings given above. In this embodiment, R₅ is preferably substituted or unsubstituted linear, branched or cyclic C₁-C₆-alkyl, aryl, arylalkyl or heteroaryl. Suitable substituents include hydroxyl groups and amino groups. In another embodiment, R₅ is the sidechain of one of the twenty naturally occurring amino acids, for example, the side chain of aspartic acid, glutamic acid, alanine, leucine, valine, asparagine, glutamine, tryptophan, threonine, arginine, cysteine, methionine, tyrosine, phenylalanine, lysine, histidine, isoleucine, or serine. Preferred identities for R₅ include amino acid side chains which are hydrophobic, such as those of valine, leucine, isoleucine, alanine, phenylalanine, methionine and tryptophan, and those which are polar but uncharged, such as asparagine, glutamine, serine, threonine, and tyrosine. In another embodiment, R₃ and R₅ together form a C₃-C₆-alkylene group. The carbon atom to which R₅ is attached is chiral and can be present in either of the two possible stereochemistries. For example, the unit —N(R₃)—CH(R₅)—C(O)— can have a configuration which is equivalent to the D- or L-configuration of an α-amino acid. In a preferred embodiment, this group has the configuration which is equivalent to the D-configuration of an α-amino acid.

In the compounds of the invention, when any of R₁-R₈ is an alkyl group, preferred alkyl groups are substituted or unsubstituted normal, branched or cyclic C₁-C₆ alkyl groups. Particularly preferred alkyl groups are normal or branched C₁-C₄ alkyl groups. A substituted alkyl group includes at least one non-hydrogen substituent, such as an amino group, an alkylamino group or a dialkylamino group; a halogen, such as a fluoro, chloro, bromo or iodo substituent; or hydroxyl.

When at least one of R₄ and R₅ is a substituted or unsubstituted aryl or heteroaryl group, preferred groups include substituted and unsubstituted phenyl, naphthyl, indolyl, imidazoly and pyridyl. When at least one of R₄ and R₅ is substituted or unsubstituted arylalkyl or heteroarylalkyl, preferred groups include substituted and unsubstituted benzyl, naphthylmethyl, indolylmethyl, imidazolylmethyl and pyridylmethyl groups. Preferred substituents on aryl, heteroaryl, arylalkyl. and heteroarylalkyl. groups are independently selected from the group consisting of amino, alkyl-substituted amino, halogens, such as fluoro, chloro, bromo and iodo; hydroxyl groups and alkyl groups, preferably normal or branched C₁-C₆-alkyl groups, most preferably methyl groups.

R₇ and R₈, in addition to alkyl, substituted alkyl or hydrogen, can each also independently be a substituted or unsubstituted azacycloalkyl group or a substituted or unsubstituted azacycloalkylalkyl group. Suitable substituted azacycloalkyl groups include azacycloalkyl groups which have an N-alkyl substituent, preferably a C₁-C₄-alkyl substituent and more preferably an N-methyl substituent. R₇ and R₈ can also, together with the nitrogen atom to which they are attached, form a heterocyclic ring system, such as a substituted or unsubstituted five or six-membered aza- or diazacycloalkyl group. Preferably, the diazacycloalkyl group includes an N-alkyl substituent, such as a C₁-C₄-alkyl substituent or, more preferably, a methyl substituent.

Specific compounds of the invention include the compounds whose structures are set forth below:

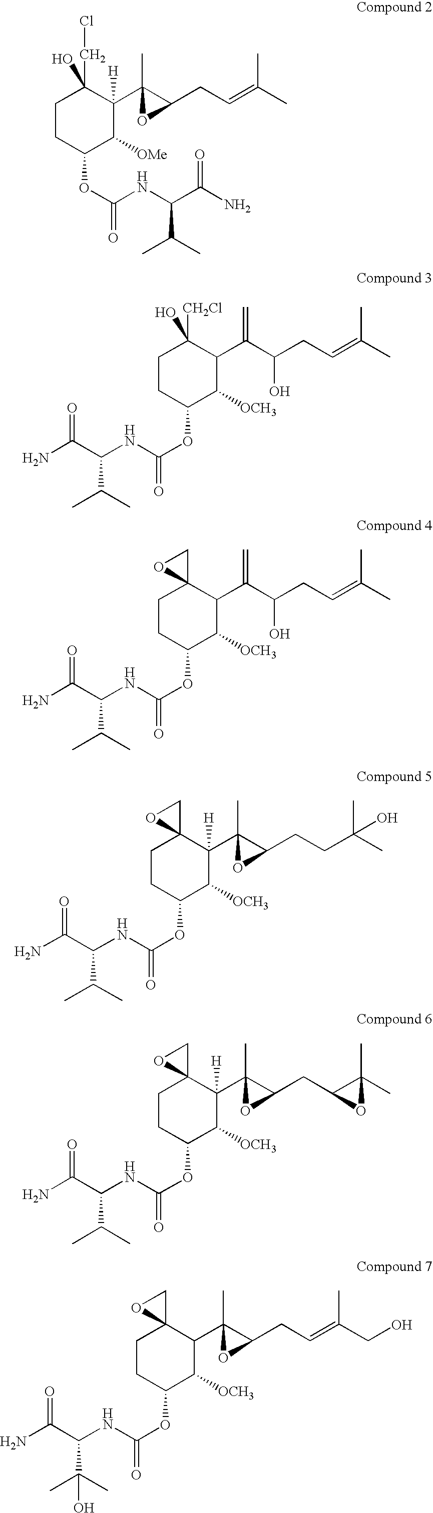

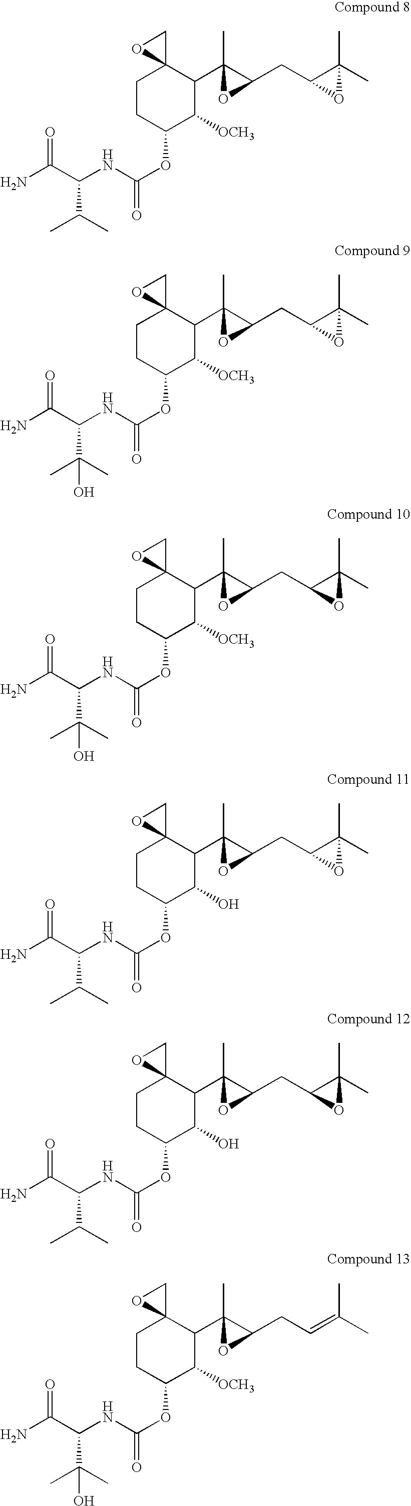

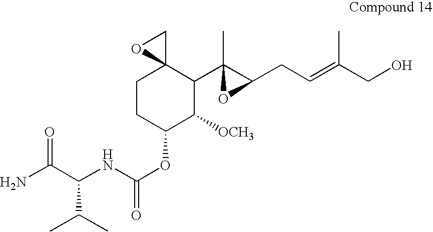

The present invention also includes salts of the compounds of the invention. Preferred salts are salts which are pharmaceutically acceptable. A “pharmaceutically acceptable salt” includes a salt that retains the desired biological activity of the parent compound and does not impart any undesired toxicological effects. Examples of such salts are salts of inorganic acids, such as hydrochloric acid, hydrobromic acid, sulfuric acid, phosphoric acid, nitric acid, and the like; and organic acids, such as acetic acid, oxalic acid, tartaric acid, succinic acid, malic acid, benzoic acid, pamoic acid, alginic acid, methanesulfonic acid, naphthalenesulfonic acid, and the like. Also included are salts of cations such as alkali and alkaline earth metals, including sodium, potassium, lithium, zinc, copper, barium, bismuth, calcium, and the like; ammonium, and organic cations such as alkylammonium, dialkylammonium, trialkylammonium and tetraalkylammonium. Combinations of the two or more of the above salts are also within the scope of the invention.

The compounds of the invention can be prepared using methods which are known in the art. As set forth in the Examples, Compound 1, below, can used as a starting material in the synthesis of certain compounds of the invention. The synthesis of Compound 1 is disclosed in U.S. Pat. No. 6,548,477, incorporated herein by reference in its entirety.

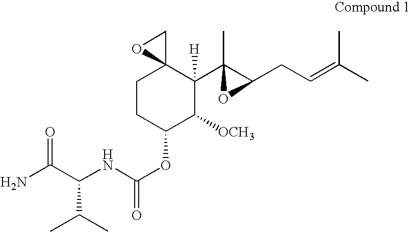

The compounds of the invention are inhibitors of the enzyme methionine aminopeptidase-2 (MetAP-2) and therefore can be used to treat a variety of diseases and conditions in which this enzyme is a therapeutic target, including those set forth in U.S. Pat. No. 6,548,477 and published PCT application WO03/092608, incorporated by reference herein in its entirety.

As one example, inhibitors of MetAP-2 inhibit endothelial cell proliferation and, therefore, exhibit anti-angiogenesis activity. Thus, the compounds of the invention can be used in a method of treating an angiogenic disease in a subject. The method includes administering to the subject a therapeutically effective amount of a compound of the present invention, thereby treating the angiogenic disease in the subject.

As used herein, the term “angiogenic disease” includes a disease, disorder, or condition characterized or caused by aberrant or unwanted, e.g., stimulated or suppressed, formation of blood vessels (angiogenesis). Aberrant or unwanted angiogenesis may either cause a particular disease directly or exacerbate an existing pathological condition. Examples of angiogenic diseases include ocular disorders, such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, ocular tumors and trachoma, and other abnormal neovascularization conditions of the eye, where neovascularization may lead to blindness; disorders affecting the skin, e.g., psoriasis and pyogenic granuloma; cancer, e.g., carcinomas and sarcomas, where progressive growth is dependent upon the continuous induction of angiogenesis by these tumor cells, lung cancer, brain cancer, kidney cancer, colon cancer, liver cancer, pancreatic cancer, stomach cancer, prostate cancer, breast cancer, ovarian cancer, cervical cancer, melanoma, and metastatic versions of any of the preceding cancers; pediatric disorders, e.g., angiofibroma, and hemophiliac joints; blood vessel diseases such as hemangiomas, and capillary proliferation within atherosclerotic plaques; disorders associated with surgery, e.g., hypertrophic scars, wound granulation and vascular adhesions; and autoimmune diseases such as rheumatoid, immune and degenerative arthritis, where new vessels in the joint may destroy articular cartilage, and scleroderma.

The term angiogenic disease also includes diseases characterized by excessive or abnormal stimulation of endothelial cells, including but not limited to intestinal adhesions, Crohn's disease, atherosclerosis, scleroderma, and hypertrophic scars, i.e., keloids; diseases that have angiogenesis as a pathologic consequence such as cat scratch disease (Rochele ninalia quintosa) and ulcers (Helicobacter pylori). In addition, the compounds of the present invention are useful as birth control agents (by virtue of their ability to inhibit the angiogenesis dependent ovulation and establishment of the placenta) and may also be used to reduce bleeding by administration to a subject prior to surgery.

The compounds of the invention can also be used to treat a subject suffering from a thymoma. Thus the invention provides a method of treating a thymoma in a patient, comprising the step of administering to the patient a therapeutically effective amount of a compound of the invention.

The compounds of the invention can also be used as immunosuppressive agents in clinical protocols in which suppression of the immune system is desired. Thus, the present invention provides a method of inducing an immunosupressed condition in a subject, comprising the step of administering to the subject an immunosupressive amount of a compound of the invention. For example, the compounds of the invention can be used to suppress immune function in subjects undergoing, or who have undergone, an organ, tissue or cell transplant from a donor. In one embodiment, the transplanted tissue, organ or cell is bone marrow, stem cells, pancreatic cells, such as islet cells, or cornea. In another embodiment, the transplanted organ is a solid organ, such as a liver, a kidney, a heart or a lung.

The compounds of the invention may also be used to treat a subject (e.g., a mammal, such as a human) suffering from a lymphoid malignancy. The method includes administering to a subject an effective amount of a MetAP-2 inhibitor, thereby treating a subject suffering from a lymphoid malignancy.

The compounds of the invention may also be used to treat rheumatic diseases, such as rheumatoid arthritis, lupus, akylosing spondylitis, psoriatic arthritis, scleroderma, Kawasaki syndrome and other rheumatic diseases as set forth in Primer on the Rheumatic Diseases, 11th Edition (John H. Klippel, MD, editor; Arthritis Foundation:Atlanta Ga. (1997)).

As used herein, the term “lymphold malignancy” includes any malignancy of a lymphoid cell. Examples of lymphoid malignancies include lymphoid leukemias, such as chronic lymphoid leukemia and acute lymphoid leukemia, and lymphomas, such as Hodgkin's disease and Non-Hodgkins lymphoma. The term “Non-Hodgkins lymphoma” includes T cell lymphomas, such as Precursor (peripheral) T-cell lymphoblastic, Adult T-cell, extranodal Natural Killer/T-cell, nasal type, enteropathy type T-cell, hepatosplenic T-cell, subcutaneous panniculitis like T-cell, skin (cutaneous) lymphomas, anaplastic large cell peripheral T-cell, and angioimmunoblastic T-cell lymphomas; and B cell lymphomas, such as precursor B lymphoblastic, small lymphocytic, B-cell prolymphocytic, lymphoplasmacytic, splenic marginal zone, extranodal marginal zone MALT, nodal marginal zone, follicular, mantle cell, diffuse large B-cell, primary mediastinal large B-cell, primary effusion and Burkitt's lymphomas. Non-Hodgkins lymphoma also includes AIDS-related lymphoma and central nervous system lymphoma.

In another aspect, the present invention provides a method of treating a subject suffering from a parasitic infection, such as an infection by a Plasmodium species, such as Plasmodium falciparum, or an infection by a Leishmania species, such as Leishmania donavani. The method comprises the step of administering to the subject a therapeutically effective amount of a compound of the invention.

In a further aspect, the invention provides a method of treating a subject suffering from an autoimmune disorder. The method includes administering to the subject a therapeutically effective amount of a compound of the invention.

As used herein, the term “autoimmune disorder” includes a disorder, disease or condition that is associated with or caused by a person's immune system attacking his or her own body. The immune system creates antibodies against its own tissues. Virtually every part of the body is susceptible to an autoimmune disorder Examples of autoimmune disorders include, but are not limited to, autoimmune hemolytic anemia, in which the immune system destroys a person's red blood cells; autoimmune hepatitis, which causes inflammation of the liver; Berger's disease, also known as IgA nephropathy, which causes kidney damage; chronic fatigue syndrome, which causes feelings of malaise, or a vague feeling of illness; Crohn's disease, which causes inflammation in the bowels; dermatomyositis, which affects the skin and muscles; fibromyalgia, which causes chronic pain and stiffness in the muscles; Graves' disease, which affects the thyroid gland; Hashimoto's thyroiditis, which is a chronic inflammation of the thyroid gland; idiopathic thrombocytopenia purpura, which causes low platelet counts and interferes with normal blood clotting; lichen planus, which affects the skin, eyes, and linings of the mouth and genitals; multiple sclerosis, in which the body attacks parts of the nervous system; myasthenia gravis, which causes severe muscle weakness; psoriasis, which causes skin lesions and itching; rheumatic fever, which causes damage to body organs, including the heart, following a strep infection; rheumatoid arthritis, in which the body attacks the joints; scleroderma, which involves the skin, gut, and other structures; Sjogren syndrome, which causes dry eyes and mouth; systemic lupus erythematosus, in which the body attacks connective tissue in joints and also in the kidneys; type 1 diabetes, a condition in which the individual doesn't produce enough insulin; ulcerative colitis, which also causes inflammation in the bowels; and vitiligo, which causes a decrease in skin pigments. In a preferred embodiment, the autoimmune disease is not rheumatoid arthritis.

As used herein, the term “parasitic infection” includes an infection caused by any parasite. Examples of parasitic infections include those caused by, for example, a Plasmodium species, such as Plasmodium falciparum, or by a Leishmania species, such as Leishmania donavani. Further examples of parasitic infections include those caused by, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazelia, Amblyospora, Ameson, Glugea, Pleistophora and Microporidium spp.; and members of the Ascetospora phylum such as, for example, Haplosporidium.

As used herein, the term “subject” includes warm-blooded animals, preferably mammals, including humans. In a preferred embodiment, the subject is a primate. In an even more preferred embodiment, the subject is a human.

As used herein, the term “administering” to a subject includes dispensing, delivering or applying an angiogenesis inhibitor compound, e.g., an angiogenesis inhibitor compound in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, eg., sufficient to treat an angiogenic disease in a subject. An effective amount of an angiogenesis inhibitor compound, as defined herein may vary according to factors such as the disease state, age, and weight of the subject, and the ability of the angiogenesis inhibitor compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

A therapeutically effective amount of a compound of the invention (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including, but not limited to, the severity of the disease or disorder, previous treatments, the general health and/or age of the subject, and other diseases present, if any. Moreover, treatment of a subject with a therapeutically effective amount of a compound of the invention can include a single treatment or, preferably, can include a series of treatments. In one example, a subject is treated with a compound of the invention in the range of between about 0.1 and 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The methods of the invention further include administering to a subject a therapeutically effective amount of an angiogenesis inhibitor compound in combination with another pharmaceutically active compound known to treat an angiogenic disease, e.g., a chemotherapeutic agent such as Taxol, Paclitaxel, or Actinomycin D, or an antidiabetic agent such as Tolbutamide; or a compound that may potentiate the activity of the compound of the invention, such as heparin or a sulfated cyclodextrin. Other pharmaceutically active compounds that may be used can be found in Harrison's Principles of Internal Medicine, Thirteenth Edition, Eds. T. R. Harrison et al. McGraw-Hill: N.Y., NY; and the Physicians Desk Reference 50th Edition 1997, Oradell, N.J., Medical Economics Co., the complete contents of which are expressly incorporated herein by reference. The compound of the invention and the other pharmaceutically active compound may be administered to the subject in the same pharmaceutical composition or in different pharmaceutical compositions (at the same time or at different times).

Pharmaceutical Compositions

The present invention also provides pharmaceutically acceptable formulations comprising one or more compounds of the invention. Such pharmaceutically acceptable formulations typically include one or more compounds of the invention as well as a pharmaceutically acceptable carrier(s) and/or excipient(s). As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat angiogenic disease, e.g., a chemotherapeutic agent such as Taxol, Paclitaxel, or Actinomycin D, or an antidiabetic agent such as Tolbutamide; or compounds that may potentiate the angiogenesis inhibitory activity of the angiogenesis inhibitor compound, such as heparin or a sulfated cyclodextrin, can also be incorporated into the compositions of the invention. Suitable pharmaceutically active compounds that may be used can be found in Harrison's Principles of Internal Medicine (supra).

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble), or dispersions and sterile powders for the extemporaneous preparation of sterile solutions or dispersions for injection. For intravenous administration, suitable carriers include physiological saline, bacteriostatic water, Cremophor EL™ (BASF, Parsippany, N.J.) or phosphate buffered saline (PBS). In all cases, the pharmaceutical composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as mannitol or sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as required, followed by filtered sterilization. Generally, dispersions are prepared by incorporating the angiogenesis inhibitor compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the angiogenesis inhibitor compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the angiogenesis inhibitor compound can be incorporated with excipients and used in the form of tablets, troches, or capsules. Oral compositions can also include an enteric coating. Oral compositions can also be prepared using a fluid carrier for use as a mouthwash, wherein the angiogenesis inhibitor compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the angiogenesis inhibitor compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the angiogenesis inhibitor compounds are formulated into ointments, salves, gels, or creams as generally known in the art.

The pharmaceutical compositions of the invention can also be prepared in the form of suppositories (e.g., with conventional suppository bases such as cocoa butter and other glycerides) or retention enemas for rectal delivery.

In one embodiment, the angiogenesis inhibitor compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044, U.S. Pat. No. 5,576,018 and U.S. Pat. No. 4,883,666, the contents of all of which are incorporated herein by reference.

The compounds of the invention can also be incorporated into pharmaceutical compositions which allow for the sustained delivery of the angiogenesis inhibitor compounds to a subject for a period of at least several weeks to a month or more. Such formulations are described in U.S. Pat. No. 5,968,895, the contents of which are incorporated herein by reference.

It is especially advantageous to formulate oral or parenteral compositions in unit dosage form for ease of administration and uniformity of dosage. Unit dosage form, as used herein, refers to physically discrete units suited as unitary dosages for the subject to be treated; each unit containing a predetermined quantity of one or more compounds of the invention calculated to produce the desired therapeutic effect in association with the required pharmaceutical carrier. The specification for the unit dosage forms of the invention are dictated by and directly dependent on the unique characteristics of the therapeutic compound and the particular therapeutic effect to be achieved, and the limitations inherent in the art of compounding such compounds for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds of the invention which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of the compounds of the invention lies preferably within a range of circulating concentrations that include the ED50 with little or no toxicity. The dosage may vary within this range depending upon the dosage form employed and the route of administration utilized. For any compounds used in the methods of the invention, the therapeutically effective dose can be estimated initially from cell culture assays. A dose may be formulated in animal models to achieve a circulating plasma concentration range that includes the IC50 (i.e., the concentration of the compound which achieves a half-maximal inhibition of symptoms) as determined in cell culture.

Such information can be used to more accurately determine useful doses in humans.

Levels in plasma may be measured, for example, by high performance liquid chromatography.

Assays for Detecting the Activity of the Compounds of the Invention

The compounds of the invention may be tested for their ability to modulate (e.g., inhibit or stimulate) angiogenesis in a variety of well known assays, e.g., the rat aortic ring angiogenesis inhibition assay or in a chorioallantoic membrane (CAM) assay. The CAM assay may be performed essentially as described in Liekens S. et al. (1997) Oncology Research 9: 173-181, the contents of which are incorporated herein by reference. Briefly, fresh fertilized eggs are incubated for 3 days at 37° C. On the third day, the shell is cracked and the egg is placed into a tissue culture plate and incubated at 38° C. For the assay, the angiogenesis inhibitor compound to be tested is attached on a matrix of collagen on a nylon mesh. The mesh is then used to cover the chorioallantoic membrane and the eggs are incubated at 37° C. If angiogenesis occurs, new capillaries form and grow through the mesh within 24 hours. The ability of the angiogenesis inhibitor compound (at various concentrations) to modulate, e.g., inhibit, angiogenesis, e.g., FGF-induced angiogenesis, may then be determined.

The compounds of the invention may also be tested for their ability to modulate (e.g., inhibit or stimulate) human endothelial cell growth. Human umbilical vein endothelial cells (HUVEC) may be isolated by perfusion of an umbilical vein with a trypsin-containing medium. HUVEC may then be cultured in GIT medium (Diago Eiyou Kagaku, Co., Japan) supplemented with 2.5% fetal bovine serum and 2.0 ng/ml of recombinant human basic fibroblast growth factor (rbFGF, Biotechnology Research Laboratories, Takeda, Osaka, Japan) at 37° C. under 5% CO₂ and 7% O₂. HUVEC are then plated on 96-well microtiter plates (Nunc, 1-67008) at a cell density of 2×10³/100 μl of medium. The following day, 100 μl of medium containing rbFGF (2 ng/ml. at the final concentration) and each angiogenesis inhibitor compound at various concentrations may be added to each well. The angiogenesis inhibitor compounds are dissolved in dimethylsulfoxide (DMSO) and then diluted with culture medium so that the final DMSO concentration does not exceed 0.25%.

After a 5-day culture, medium is removed, 100 μl of 1 mg/ml of MTT (3-(4,5-dimethyl2-thiazolyl)-2,5-diphenyl-2H-tetrazolium bromide) solution is added to the wells, and microtiters are kept at 37° C. for 4 hours. Then, 100 μl of 10% sodium dodecyl sulfate (SDS) solution is added to wells, and the microtiters are kept at 37° C. for 5 to 6 hours. To determine the effects of the angiogenesis inhibitor compound on cell number, the optical density of each well at 590 nm is measured using an optical densitometer.

The ability of the angiogenesis inhibitor compounds of the invention to modulate capillary endothelial cell migration in vitro may also be tested using the Boyden chamber assay (as described in Falk et al. (1980) J Immunol. Meth. 33:239-247, the contents of which are incorporated herein by reference). Briefly, bovine capillary endothelial cells are plated at 1.5×10⁴ cells per well in serum-free DMEM (Dulbecco's Modified Eagle's Medium) on one side of nucleopore filters pre-coated with fibronectin (7.3 μg fibronectin/ml PBS). An angiogenesis inhibitor compound is dissolved in ethanol and diluted in DMEM so that the final concentration of ethanol does not exceed 0.01%. Cells are exposed to endothelial mitogen (Biomedical Technologies, Mass.) at 200 μg/ml and different concentrations of the angiogenesis inhibitor compound in serum-free DMEM for 4 hours at 37° C. At the end of this incubation, the number of cells that migrate through 8 micron pores in the filters is determined by counting cells with an ocular grid at 100× in quadruplicate.

The ability of the compounds of the invention to modulate tumor growth may be tested in vivo. An animal model, e.g., a C57BL/6N mouse with a mouse reticulum cell sarcoma (M 5076) intraperitoneally transplanted therein, may be used. The tumor cells in ascites can be collected by centrifugation, and suspended in saline. The cell suspension (2×10⁶ cells/100 μl/mouse) is inoculated into the right flanks of mice. Tumor-bearing mice are then subcutaneously treated with the test compound (at various concentrations suspended in 5% arabic gum solution containing 1% of ethanol) for 12 days beginning one day after the tumor inoculation.

The tumor growth may be determined by measuring tumor size in two directions with calipers at intervals of a few days.

Finally, the ability of the compounds of the invention to modulate the activity of MetAP2 may be tested as follows. Recombinant human MetAP2 may be expressed and purified from insect cells as described in Li and Chang, (1996) Biochem. Biophys. Res. Commun. 227:152. Various amounts of test compound is then added to buffer H (10 mM Hepes, pH 7.35, 100 mM KCl, 10% glycerol, and 0.1 M Co²⁺) containing 1 nM purified recombinant human MetAP2 and incubated at 37° C. for 30 minutes. To start the enzymatic reaction a peptide containing a methionine residue, e.g., Met-Gly-Met, is added to the reaction mixture (to a concentration of 1 mM). Released methionine is subsequently quantified at different time points (e.g., at 0, 2, 3, and 5 minutes) using the method of Zou et al. (1995) Mol. Gen. Genetics 246:247-253).

This invention is further illustrated by the following examples which should not be construed as limiting. The contents of all references, patents and published patent applications cited throughout this application, as well as the Figures are hereby incorporated by reference.

Various aspects of the invention are described in further detail in the following subsections.

### EXAMPLES

### Example 1

### Synthesis of Compound 2

Compound 1 was synthesized as set forth in Example 5 of U.S. Pat. No. 6,548,477, the teachings of which are hereby incorporated herein by reference in their entirety. Compound 1 (1.0 g, 2.36 mmole) was dissolved in 20 mL 1,4 dioxane. To the stirred solution was added 4.0 M HCl in dioxane (0.65 mL, 2.59 mmole, 1.1 equiv.), and the reaction was stirred for a further 15 min., after which it was concentrated in vacuo. It was then lyophilized from 20% acetonitrile in water, and purified by reverse phase preparative HPLC using an acetonitrile-water gradient.

### Example 2

### Synthesis of Compound 3

Compound 1 (502 mg, 1.2 mmole) was dissolved in 10 mL 1,4 dioxane in a nitrogen flushed, 50 mL round bottom flask. To the stirred solution was added 4.0 M HCl in dioxane (0.73 mL, 2.92 mmole, 2.5 equiv.), and the reaction was stirred for a further 2 h., at which time LC-MS showed complete disappearance of starting material. The reaction mixture was concentrated in vacuo to a thick, white oil which was sufficiently pure for conversion into Compound 3. Alternatively, it could be purified by reverse phase, preparative HPLC using an acetonitrile-water gradient.

### Example 3

### Synthesis of Compound 4

Compound 3 (500 mg, 1.2 mmole) was dissolved in 8.0 mL dry THF in a nitrogen flushed, 50 mL round bottom flask. Potassium t-butoxide (251 mg, 2.3 mmole) was added and the reaction mixture stirred for one hour, at which time LC-MS showed complete disappearance of starting material. The reaction mixture was concentrated at reduced pressure and resuspended in dichloromethane. The organic layer was washed with 2× saturated sodium bicarbonate, 2× water, and 2× brine, and then dried over sodium sulfate and concentrated to a clear, thick oil. It was purified by reversed phase, preparative HPLC using an acetonitrile-water gradient.

### Example 4

### Synthesis of Compound 5

Mercury(II) acetate (319 mg, 1.0 mmole) was dissolved in 1 mL water. THF (1 mL) was added, forming a yellow-orange suspension. After stirring at room temperature for 5 min., Compound 1 (424 mg, 1.0 mmole) was added in one portion. The solution immediately clarified. After stirring for 1 h. at room temperature, the reaction was chilled in an ice bath, and to it was added 3 N NaOH (1 mL), followed by sodium borohydride (19 mg, 0.5 mmole, 2 equiv.) dissolved in 1 mL of 3 N NaOH. Following another hour of stirring, the reaction mixture was allowed to stand overnight in a separatory funnel to facilitate removal of precipitated mercury. Brine was added to the aqueous layer, which was extracted twice with ether. The combined organic extracts were then washed with brine, dried over MgSO₄ and concentrated to a white foam. The product was purified by reversed phase, preparative HPLC using an acetonitrile-water gradient.

### Example 5

### Inhibition of Proliferation of Endothelial Cells

A set of compounds of the invention were tested for their ability to modulate human endothelial cell growth. For this assay, human umbilical vein endothelial cells (HUVEC) were maintained in Clonetics endothelial growth medium (EGM) in a 37° C. humidified incubator. Cells were detached with trypsin and pelleted by centrifugation at 300×g for 5 minutes at room temperature. HUVEC were added to 96-well plates at 5,000 cells/well. After incubating for 6 hours, the medium was replaced with 0.2 ml fresh EGM supplemented with 0.5 nM bFGF and the desired concentration of test compound. Test compounds were initially dissolved in ethanol at stock concentrations of either 10 mM or 0.1 mM, and subsequently diluted in EGM to obtain concentrations from 1 pM to 10 μM. After 48 hours at 37° C., the medium was replaced with fresh bFGF-supplemented EGM and test compound. Following incubation for an additional 48 hours at 37° C., MTT (3-[4,5-dimethylthiazol yl]-2,5-diphenyl-tetrazolium bromide) was added to 1 mg/ml. After 2-4 hours at 37° C. the medium was replaced with 0.1 ml/well isopropanol. The plates were placed on a shaker for 15 minutes at room temperature and analyzed in a Labsystems Multiskan plate spectrophotometer to determine the optical density at 570 nm.

The results of the assays, set forth in FIG. 1, demonstrate that the compounds of the invention are able to inhibit endothelial cell growth at nanomolar concentrations.

Equivalents

Those skilled in the art will recognize, or be able to ascertain using no more that routine experimentation, many equivalents to the specific embodiments of the invention described herein. Such equivalents are intended to be encompassed by the following claims.

1. A compound of the formula A-B wherein
A is a moiety selected from the group consisting of 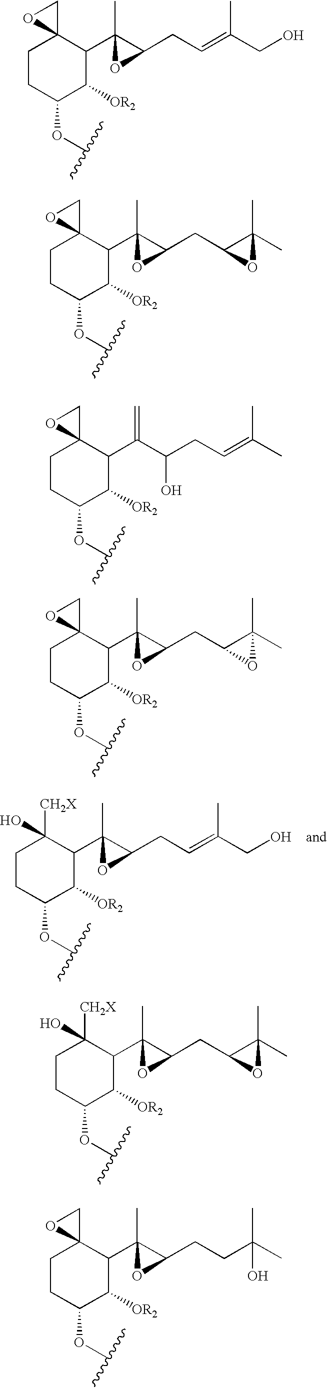
wherein R₂ is hydrogen or C₁-C₆-alkyl and X is halogen, dialkylsulfinium, thioalkoxy or thioaryloxy; and
B is an alkanoyl, aroyl, carbamoyl or substituted carbamoyl group;
or a pharmaceutically acceptable salt thereof.

2. The compound of claim 1 wherein B is a moiety of the formula 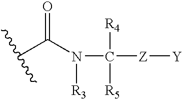
wherein
R₃ is hydrogen or alkyl;
R₄ and R₅ are each, independently, hydrogen, substituted or unsubstituted alkyl, substituted or unsubstituted aryl, substituted or unsubstituted arylalkyl, substituted or unsubstituted heteroaryl or substituted or unsubstituted heteroalkyl; or
R₃ and R₅ together form an alkylene group;
Z is —C(O)— or -alkylene-C(O)—; and
P is —OR₆ or —N(R₇)R₈, wherein R₆, R₇ and R₈ are each, independently, hydrogen, substituted or unsubstituted alkyl, substituted or unsubstituted aryl or substituted or unsubstituted azacycloalkyl or R₇ and R₈, together with the nitrogen atom to which they are attached, form a heterocyclic ring structure.

3. The compound of claim 1 wherein B is a moiety of the structure 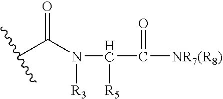
wherein R₅ is substituted or unsubstituted linear, branched or cyclic C₁-C₆-alkyl, aryl, arylalkyl or heteroaryl; or R₃ and R₅ together form a C₃-C₆-alkylene group.

4. The compound of claim 3 wherein
R₅ is linear or branched C₁-C₆-alkyl, hydroxyl-substituted linear or branched C₁-C₆-alkyl; and
R₃, R₇ and R₈ are each hydrogen.

5. A compound selected from the group consisting of 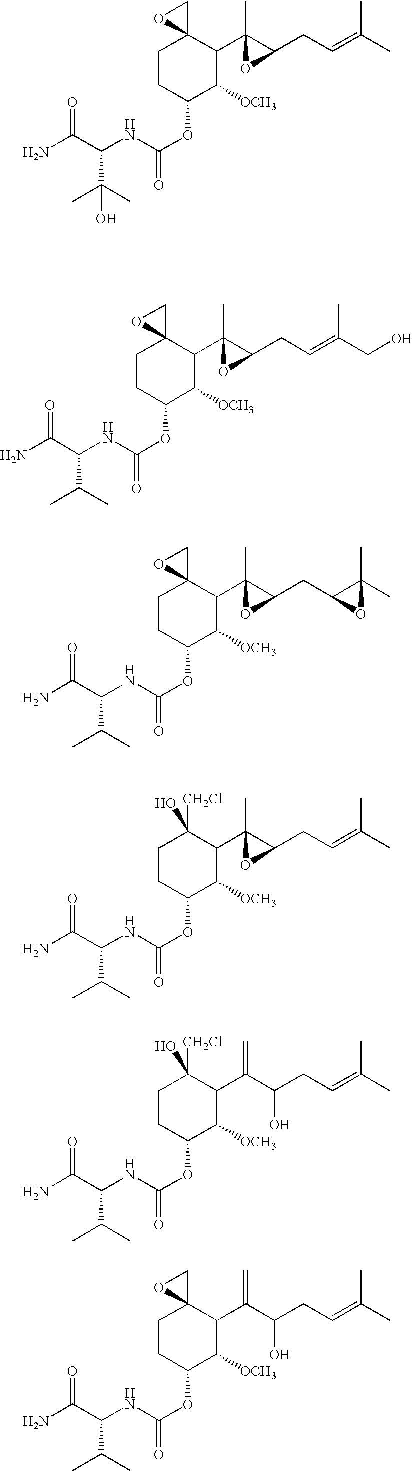 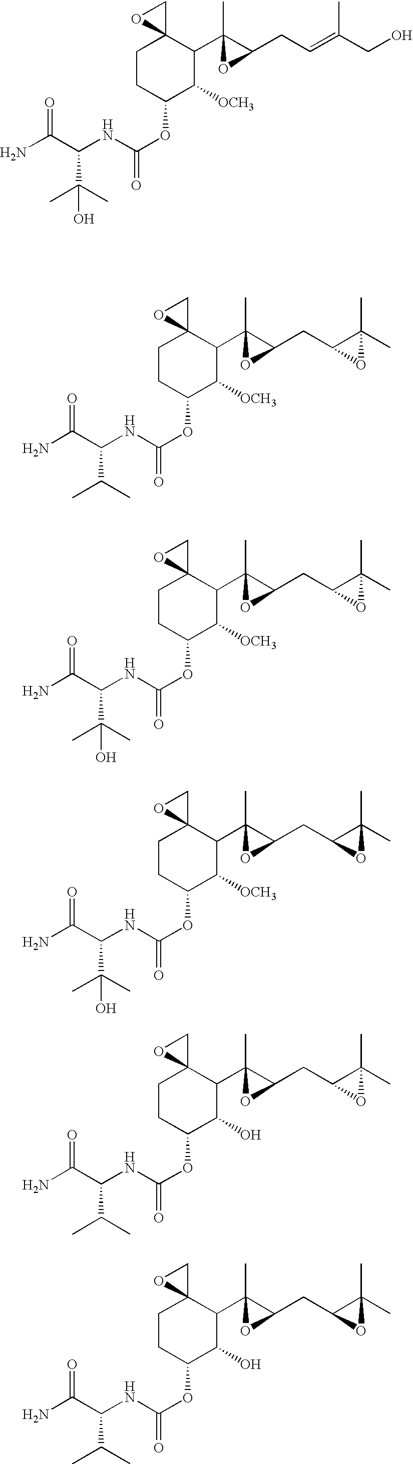
and

6. A pharmaceutical composition comprising a compound of claim 1 or 2 and a pharmaceutically acceptable carrier.

7. A method of treating an angiogenic disease in a subject, comprising the step of administering to the subject a therapeutically effective amount of a compound of claim 1 or 2.

8. The method of claim 7 wherein the angiogenic disease is cancer.

9. The method of claim 8 wherein the angiogenic disease is lymphoma.

10. A method of treating an autoimmune disease in a subject, comprising the step of administering to the subject a therapeutically effective amount of a compound of claim 1 or 2.

11. The method of claim 10 wherein the autoimmune disease is rheumatoid arthritis, psoriasis or multiple sclerosis.

12. A method of treating a parasitic infection in a subject, comprising the step of administering to the subject a therapeutically effective amount of a compound of claim 1 or 2.

13. The method of claim 12 wherein the infection is by a parasite selected from the group consisting of Plasmodium species and Leishmania speicies.
